(19) United States
(12) Patent Application Publication
(10) Pub. No.: US 2011/0295062 A1
(43) Pub. Date: Dec. 1, 2011

(54) EQUIPMENT FOR INFRARED VISION OF ANATOMICAL STRUCTURES AND SIGNAL PROCESSING METHODS THEREOF

(75) Inventors: Eduard Gratacós Solsona, Barcelona (ES); Iván Amat Roldán, Barcelona (ES)

(73) Assignees: Fundacio Clinic Per A La Recerca Biomedica Fcrb, Barcelona (ES); Universitat De Barcelona, Barcelona (ES); Hospital Clinic I Provincial De Barcelona, Barcelona (ES)

(21) Appl. No.: 13/139,210

(22) PCT Filed: Dec. 10, 2009
(86) PCT No.: PCT/EP2009/066799

§ 371 Date: Aug. 9, 2011

(30) Foreign Application Priority Data
Dec. 11, 2008 (ES) 200803513 ES

(51) Int. Cl. A61B 1/04

(52) U.S. Cl. 600/109

(57) ABSTRACT

Equipment for infrared vision of anatomical structures applicable to assist the physicians in endoscopic, fetoscopic or laparoscopic operations and methods for signal processing to enhance said vision, comprising two units that work together: a multimodal or multispectral imaging unit, constituted by a device comprising an endoscope or fetoscope or laparoscope, and additional optical systems to acquire multimodal images of the interior of the patient's body; and an image processing unit, to which said images are transferred, comprising processing devices with a navigation interface which process said images and display the patient's enhanced anatomical map image and the endoscope location, equipped with hardware and software that apply at least five different vision-enhancing methods, namely normalization, segmentation, tracking, mapping and fusion.

### FIELD OF THE INVENTION

The present invention relates to the field of photonics, image acquisition, image processing, vision enhancement and information extraction applied to life sciences, mainly focused on the medical and biomedical fields and specially, but not exclusively, on the fields of endoscopy, fetoscopy and laparoscopy.

More specifically, the invention relates to a medical device or equipment comprising means for acquiring multimodal or multispectral images of a living subject, including illumination means and a digital image processing platform associated to said means, with embedded algorithms to extract and/or enhance specific image information with the objective of assisting physicians in their decisions, for example, when diagnosing, monitoring and/or performing a given therapy or surgical operation.

### BACKGROUND OF THE INVENTION

In the medical field a number of imaging systems have been devised to display and enhance the visualization of an area under examination, either with diagnostic purposes or to guide therapeutic surgical procedures. It is apparent that any relevant information of the area under inspection plays an important role in the clinical assessment of the subject's condition which is of major importance when making clinical decisions that may ultimately affect the well being and quality of life of the patient. This is of critical importance for instance, in endoscopic surgery, where an accurate vision is essential for the results of the operation. In general, the visualization of critical structures (i.e. blood vessels and nerves) involves three typical situations: (i) said structures cannot be distinguished due to poor visualization conditions, (ii) the structures are hidden beneath a layer of other tissue and/or (iii) the structure of interest is not distinguishable from the surrounding structures. Particularly relevant to this field is the task of enhancing and/or generating image contrast and improving the definition of the visualization of blood vessels and/or different functional aspects about these vessels, which are poorly or non visible by endoscopy with the presently available technology without using an exogenous contrast agent.

Despite endoscopy is an advanced surgical technique which greatly minimizes surgical procedure risks, some problems still persist, like the risk of accidentally cutting a mayor blood vessel. Even though the use of minimally invasive endoscopy offers clear advantages to the patient, it imposes certain disadvantages to the physicians, like a constrained vision of the surgical field or a poor contrast and/or definition. The need to accurately identify blood vessels under such conditions may represent a serious challenge to any surgeon, rendering surgery extremely dependent on the surgeon's experience, resulting in prolonged operations due to bleeding episodes, and occasionally resulting in major hemorrhagic complications.

A number of inventions have been devised to improve medical imaging relating to the visualization of blood vessels. Some of the previous inventions refer to specific solutions for problems in the above-discussed field. For instance there are several patents describing particular lighting systems, like U.S. Pat. Nos. 7,041,054 or 6,730,019. These patents describe illumination and image acquisition systems with particular preferred embodiments. It should be noted that the present invention does not rely on a particular illumination method. Although illumination is needed in the system, the system is independent of the method employed to achieve that illumination. Moreover, the system is able to cope with and adapt to different illumination schemes to achieve its purpose, that is, to enhance and/or generate image contrast and improve definition for the visualization of anatomical structures. Other inventions relate to a particular method for capturing the images or disclose embodiments of specific image capturing devices, like US 2008/0208006. Again as with the illumination, the present invention is independent of the method of capturing the image; it can be used according to different image capturing approaches.

U.S. Pat. No. 5,255,087 by Olympus describes a video system comprising a well detailed lighting system used in combination with a standard endoscope, a control system and an image capturing and processing unit. The goal of said system is to improve the images of an endoscopic system. In order to achieve this goal three techniques are described by the inventor: auto fluorescence imaging (AFI), narrow band imaging (NBI) and Infrared Imaging (IRI). AFI is based on the principle of the auto fluorescence of certain tissues; NBI is based on a well know technique of using contrast agents and an illumination of a particular wavelength to which the contrast agent is sensitive; and IRI is a specific combination of the two previous techniques which uses an exogenous contrast agent like indocyanine green (ICG) to detect submucosal blood vessels, but it is used only for diagnostic purposes. That limitation to a purely diagnostic use is because the injected dye rapidly dissolves into the bloodstream, and the five minutes that it lasts would not allow using it in therapy or surgery, which require much longer duration. However, another disadvantage with respect to the present invention is that it requires a contrast agent to enhance the visualization of blood vessels, both superficial (that is apparent to the naked eye) and submucosal (running under the mucosa and therefore normally non visible to simple inspection), whereas the present invention makes use of an algorithm to perform such feature, making it less invasive and hence more appropriate in the surgery field. In addition to the fact that IRI is not designed as an assistance vision system for surgery, the said technique does not provide other additional features like: image segmentation, image mapping of the surgical field, or functional assessment of blood vessels (by obtaining relevant information such as, for example, the amount of oxygen carried by the blood or the coagulation state of the vessels). Those features are supplied by the present invention, and are differential and provide useful information when used for surgical endoscopic procedures, including laparoscopy or fetoscopy. In those surgical techniques the availability of a complete vascular map of the surgical field or the capability to distinguish the coagulation status of a vessel, might represent extremely valuable information to assist the surgeon during the operation.

Patent US 2005/0182321 discloses a similar invention as the one previously commented concerning IRI, based on a medical imaging enhancing system using visible and infrared images in combination with a dye agent. A mayor disadvantage of this system compared with the present invention is that it requires of a contrasting agent or dye to be injected to the blood stream of the patient, consequently rendering this system non-usable in any surgical procedure for the reasons above mentioned, namely the rapid dilution of the contrast agent into the bloodstream with the consequent inefficiency to assist physicians in carrying out therapy or surgery. Additionally, said patent only contemplates the use of a visible and a single near infrared (NIR) channel, constraining the image capturing process to a total of four spectral bands without mentioning the possible use of more NIR channels or other imaging modes that could improve the detection of vessels and the extraction of vessel functional information. A further weakness is that it only focuses on the ability of detecting blood vessels and does not provide specific embedded methods as, for example, image segmenting, image mapping and assessing vessel functionality.

Patent US 2008/0097225 explicitly mentions specific optical techniques, namely optical coherence tomography (OCT) and spectrally-encoded endoscopy (SSE), with the aim to reduce the size of the endoscope and increase its resolution. A significant disadvantage of said techniques is their technical complexity, since they necessary comprise a scanning unit and a complex optical assembly. Although the said patent mentions that the wavelength can be chosen to assess the amount of oxygen carried by the blood, it does not take into account the use of this information as an integrated tool for assisting the surgeon or the physician by means of enhancing the images displayed. A further disadvantage, due to the small field of view of such small instrument, is that the physician's angle of vision is substantially restricted, thus limiting considerably the feasibility of such system for surgical applications. These problems are overcome with the present invention.

A similar case is patent EP 1,839,561, which discloses an endoscopic apparatus that is a combination of a standard visible endoscopy in conjunction with an OCT arrangement, the said apparatus being a particular solution to apply to a known optical technology in a particular way. A disadvantage of said apparatus is that it can only obtain information to generate an enhanced image for a narrow portion of the area under study; furthermore it does not compose a substantially enhanced image. The said invention does not seem to have optimum use for blood vessel enhanced imaging.

U.S. Pat. No. 6,353,753 describes a device for the acquisition of images from deep anatomical structures. The main disadvantage of said device is that it does not include specific image analysis processing intended for segmenting and displaying the information in conjunction with the visual image; it also lacks image reconstruction functions.

Regarding the state of the art related to image enhancement through the capture of visible and infrared light, some advances have been achieved as, for example, in patent US 2008/0079807, where a multispectral imaging system is described with special emphasis in charge couple devices (CODs) and optoelectronics. In this case that patent focuses particularly on the image collection device technology. Major drawbacks of said patent in comparison with the present invention include the lack of tracking (defined as a signal processing procedure for tracking and localizing of blood vessels between consecutive scenes from the images generated), lack of image mapping, lack of functional analysis such as the analysis of tissue (blood vessel) oxygenation or the lack of use of more than one infrared spectral band.

The state of the art in image processing and segmentation describes various algorithms to enhance medical images and to segment certain tissues. However, the state of the art also comprises real-time platforms of different natures such as graphic processing units (GPUs), field-programmable gate arrays (FPGAs) or systems based on central processing units (CPUs). Although said algorithms and platforms describe general approaches for medical image processing that are intended to be used in the same applications of the present invention, none of them refers to an intergrated real-time tool that performs the function of the current invention. Precisely the systems disclosed in the state of the art do not combine the image processing techniques with optical illumination and image capturing/processing techniques, without the need of using contrast agents. Additionally, those other state of the art algorithms and platforms are not intended for the service of physicians with the aim of assisting surgical procedures in real time.

To sum up, prior art disclosures focus on solving specific technical problems in the field of image illumination and image capture, but do not explicitly include the function of image processing and enhancing, anatomical structures image segmentation and large-area composition using multispectral and multimodal input signals.

There remains, thus, a need for enhancing imaging systems capable of generating a better image of anatomical structures and/or blood vessels in terms of quality, contrast and information provided to the physician, without the use of contrast agents, with the ability to clearly highlight the presence of even tiny or submucosal vessels, hidden to the naked eye. Besides, the methods disclosed in the state of the art do not allow to map large vascular areas of interest, and to obtain functional information about vessels which can assist in decision during an operation. One of the objects of the present invention is to provide a new form of imaging system for endoscopic surgery that overcomes the limitations of the presently available technology.

Throughout the text, the term multimodal designates the use of more than one image acquisition method in different bands with the application of different optical techniques such as, for instance, the acquisition of red, green and blue (RGB) and NIR images, in combination with the application of polarizing filters, optical filters, digital filters, digital image processing algorithms, polarization imaging, multiphoton imaging, laser speckle imaging, dynamic speckle imaging, optical coherence tomography, two photon fluorescence, harmonic generation, optoacustics, coherent anti-Stokes Raman spectroscopy (CARS) and/or other optical elements and techniques that can generate contrast in the image generation. The term multispectral refers to the use and detection of more than one spectral band such as, for instance, RGB detection in combination with NIR detection.

### SUMMARY OF THE INVENTION

The scope of the present invention lays on the industrial sector dedicated to the manufacture of medical devices in general and in particular to robotized equipment and devices with audio visual and computerized tools. The invention is intended to assist or as guidance of physicians during medical procedures and surgical operations.

More specifically, the present invention relates to an equipment for infrared enhanced vision of anatomical structures, applicable to assist physicians during endoscopic, fetoscopic or laparoscopic procedures and/or treatments and the methods to improve said vision. The equipment and methods disclosed by the present invention constitute a novelty in this field which provides remarkable improvements and innovative features that surpass the systems currently known for the same purpose, being adequately reflected in the characterizing features that distinguish the said invention from the state of the art in the claims accompanying this technical description.

One aim of the present invention is to solve the technical difficulties that currently exist in the surgery for complications of monochorionic twins pregnancies, in order to locate and identify blood vessels coagulated by the use of a laser source for therapeutic purposes, achieving improved safety and repeatability in such surgical operations.

Other possible applications of the invention include any type of endoscopy surgery, such as gastrointestinal tract endoscopy, respiratory tract endoscopy, arthroscopy, gynecologic endoscopy, colposcopy, urologic endoscopy, otoscopy, plastic surgery endoscopy or a wide range of other medical procedures, such as skin or open surgical procedures, among others.

Other possible applications of the invention include the use of the disclosed invention in conjunction with robotized surgery. It is of relevant interest for the automated, semi-automated or remote equipment to perform surgery as the presented methods are particularly suitable to be used automated, semi-automated or remote equipment to perform surgery as they provide objective and quantitative data and reproducible behavior.

One object of the invention is an equipment designed to assist the guidance of surgical operations by means of the representation of the surgical site and its surroundings, being composed of two basic units that work together:
a multimodal or multispectral image acquisition unit, comprising an image capturing device, preferably an endoscopic image acquisition device comprising an endoscope, a fetoscope or a laparoscope and additional optical systems, to acquire multimodal or multispectral images from the interior of the human body, transferring said images to an enhanced imaging unit.
an image processing unit, wherein said unit comprises an image-processing device with a navigation interface responsible for processing and displaying the enhanced images of the human body, preferably the vascular map of the human body, and the location of the endoscope to the surgeon. For this purpose, the specific hardware and software constituting this unit and implemented in GPUs, FPGAs, CPU-based systems or any other hardware performing real-time processing through local, distributed or parallel computing, comprises at least five signal processing methods consisting essentially of:

1. Normalization: Signal processing method to normalize the amount of light that illuminates the tissue, by real-time comparing of the intensities in each of the image points of the visible light (red, green and blue) and infrared light with the intensities obtained by the application of a spatial low-pass filter implementing image-blurring functions on the images. By this method the amount of incident infrared light is estimated in a reproducible manner.

2. Segmentation: Signal processing method to segment the images of the anatomical structures or tissues, preferably vascular structures such as blood vessels, based on the real-time multimodal analysis of infrared and visible light.

3. Tracking: Signal processing method for real-time tracking and co-localizing of the anatomical structures or tissues, preferably the vascular structures such as blood vessels between two consecutive images from images generated by previous methods (normalization and segmentation).

4. Mapping: Signal processing method to generate the real-time map of the anatomical structures or tissues, preferably the vascular structures from individual images and the tracking coordinates obtained by normalization and segmentation.

5. Fusion: Signal processing method to fuse in real-time the visible image (produced by a standard endoscope) with information obtained after the mapping step.

The ability to navigate or to view vascular characteristics is greatly improved by means of the present invention since the surgeon, in addition to the standard obtained visualization, has at least the above-referred five options or new ways of visualization.

A further object of the invention is an equipment for infrared-enhanced imaging of anatomical structures and tissues, preferably vascular structures, to assist in endoscopic, fetoscopic and laparoscopic surgery, where the multimodal image acquisition unit comprises an endoscope, a fetoscope or a laparoscope with at least one channel from where the video images from inside the human body are acquired, to which an infrared light source and a white light source (or comprising at least light in blue, green and red wavelengths) are coupled. That source of light is coupled to the video channel of the endoscope by using different optical elements such as beam splitters, hot mirrors, cold mirrors, dichroic mirrors, polarizers, diffusers, diffractive optical elements, analyzers, holographic optical elements, phase plates, acusto-optic materials, dazzlers, shapers, partial mirrors, dichroic prism systems, tunable optical filters, multibifurcated light guides, polarization beam splitters or any other optical devices able to modify their transmission or reflection conditions depending on the wavelength, polarization or other optical property in order to split or combine the optical path for either or both detection and illumination, also including the encapsulation in optical fiber when the optical path is a fiber optic path.

A further object of the invention is an equipment wherein the same channel in the endoscope, fetoscope or laparoscope may be employed for the detection by using elements such as hot mirrors or optical fibers with embedded built-in mirrors (encapsulated mirrors); and where the use of additional optical elements such as filters and lenses is also envisaged, in order to form images in one or more video cameras, like for example a charge couple device, a complementary metal oxide semiconductor (CMOS) or an electron-multiplying charge couple device (EM-CCD) camera, etc., digitizing said images for later processing by the image processing unit.

Another object of the invention is an equipment where, in case that the signals detected are very weak or they possess a low quality, image intensifiers are provided to the video cameras.

A further object of the invention is an equipment wherein, alternatively and with the aim to simplify the multimodal images, the light sources are coupled to the video systems by using different channels of the endoscope. The use of more than one channel allows the definition of different light paths, thus simplifying the employment of optical elements in each channel.

A further object of the invention is an equipment wherein, alternatively, at least one channel in the endoscope, fetoscope or laparoscope is used only for the illumination in combination with optical elements; and at least one other channel is used only for the detection, wherein the equipment optionally further comprises additional optical elements such as filters and lenses.

A further object of the invention is an equipment wherein a CCD, CMOS or EM-CCD camera is installed at the probe of the endoscope and coupled to an electric connection for the detection of different bands or wavelengths sequentially emitted by light sources, wherein at least one filter in the camera can optionally be a color filter array (CFA) or a color filter mosaic (CFM) for the separation of one or more infrared spectral bands.

A further object of the invention is an equipment wherein the image acquisition unit comprises as image capturing device an optical objective adapted to skin and open surgical procedures.

A further object of the invention is a procedure of signal processing of images of anatomical structures and tissues, preferably vascular structures such as blood vessels, comprising at least five signal processing methods.

A further object of the invention is an image processing unit comprising at least five signal processing methods.

A further object of the invention is the use of an equipment, a procedure or an image processing unit in endoscopy, fetoscopy or laparoscopy.

A further object of the invention is the use of an equipment, a procedure or an image processing unit in treatments of monochorionic twins pregnancies.

A further object of the invention is the use of an equipment, a procedure or an image processing unit applied to endoscopy surgery procedures, such as gastrointestinal tract endoscopy, respiratory tract endoscopy, arthroscopy, gynecologic endoscopy, colposcopy, urologic endoscopy, otoscopy, or plastic surgery endoscopy, among others.

A further object of the invention is the use of an equipment, a procedure or an image processing unit for infrared-enhanced imaging of anatomical structures applied to skin and open surgical procedures by the replacement of the endoscope, laparoscope or fetoscope by an optical objective adapted to its application in said procedures.

A further object of the invention is the use of an equipment, a procedure or an image processing unit in order to report functional information on the anatomical structures such as the amount of oxygen level in tissues or vessels to distinguish between arteries and veins, or to assess the collagen structure of the tissues.

It is important to highlight that the system has the advantage that it does not need contrast agents to carry out the task of representing the vascular map, being that feature an essential property to perform foetal surgery (avoiding the use of substances potentially dangerous for the fetus when administered in a considerable amount or during a long period of time) and reducing, in general, the invasiveness of the rest of the surgical procedures.

Additionally, the equipment of the invention includes a device that generates a global map of the patient vascular surgical sites; in particular, in operations of complications in monochorionic twins pregnancies it facilitates viewing the vasculature of the placenta thus achieving a better surgeon's orientation.

The equipment of the invention is also able to report functional information on the anatomical structures giving an enhanced view with rich and relevant data of the field that is being imaged with not only spatial or temporal dependent information but also with information on the functional performance of the anatomical structure such amount of oxygen level in tissues or vessels and to distinguish between arteries and veins amongst others.

### DESCRIPTION OF THE FIGURES

To complete the current description and in order to better understand the features of the invention here described, a set of drawings with illustrative, but not restrictive, purpose is presented:

FIG. 1—Block diagram with the schematic representation of a preferred embodiment of the multimodal image acquisition unit integrated on the equipment of the invention, to appreciate their key elements and the interrelationship between them.

FIG. 2—Block diagram of an alternative embodiment of the multimodal image acquisition unit, in this case including two video channels for the endoscope.

FIG. 3—Block diagram of an alternative embodiment of the multimodal image acquisition unit, in this case including a video channel and an illumination channel.

FIG. 4—Block diagram of an alternative embodiment of the multimodal image acquisition unit wherein a CCD, CMOS or EM-CCD camera is installed at the probe of the endoscope and coupled to an electric connection for the detection of different bands or wavelengths sequentially emitted by the light sources.

FIG. 5—Diagram of the image processing unit built-in the equipment of the invention, to appreciate the main elements comprised therein, and the arrangement and relationship between them.

FIG. 6—(a) Local imaging obtained by the equipment described by the present invention coupled to a standard endoscope or festoscope; (b) surface vessel NIR detection; (c) digital superposition of (a) and (b); (d) detection and reconstruction of the vascular map after manual scanning by the surgeon during the operation; (e) digital superposition and mosaicing of the vascular map.

FIG. 7—Images obtained by the application of the techniques described by the present invention to the detection of vessels over the forearm's surface, by fusing visible modes with NIR images.

### DETAILED DESCRIPTION OF THE INVENTION

In view of the aforementioned figures and according to the numbering adopted in them, different embodiments of the invention are described hereunder.

Thus, as shown in said figures, the equipment comprises a multimodal image acquisition unit (1) and an image processing unit (2).

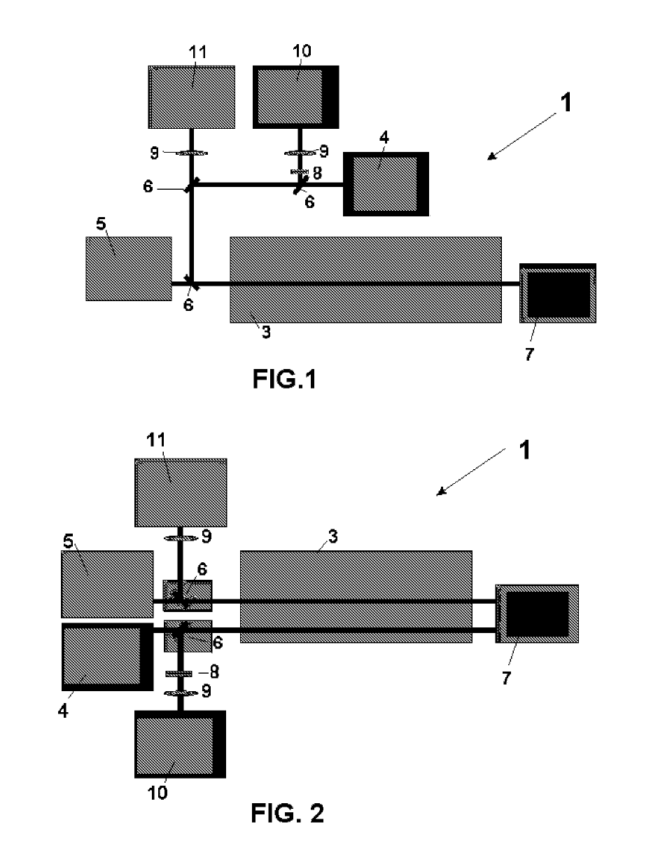

The multimodal image acquisition unit (1), whose preferred implementation as shown in FIG. 1 includes an image capturing device, preferably an endoscopic image acquisition device comprising an endoscope, a fetoscope or a laparoscope and additional optical systems, comprising said systems at least one channel from which the video images from the inside of the patient are acquired, and at least one light source to illuminate the observed tissues.

In a preferred embodiment of the invention, the video channel or channels that are available on the endoscope are coupled to an infrared light source (4) and a white light source (5) or a light source that contain at least three wavelengths within the blue, green and red.

The infrared light source (4) is, preferably:
A source belonging to the NIR (ranging from 750 nm to 1600 nm).
A source ranging from 800 nm to 900 nm.
A source ranging from 1050 to 1150 nm.
A monochromatic source centered at a wavelength between 800 and 900 nm.
A monochromatic source centered at a wavelength between 1050 and 1150 nm.
A laser Nd: YAG source (centered at 1064 nm).
A source based on titanium-sapphire laser (Ti: Sap), focusing on 700 nm to 1100 nm.
Ytterbio based laser source (Yb: KYW, Yb: KGW, etc.)
Ytterbio laser source based on Chromium, Cr: Forsterite 1230 to 1270 nm.
An infrared source based on parametric conversion methods (Optical Parametric Oscillators, Optical Parametric Amplifiers, Nonlinear Crystals, etc.).
Lights or LEDs with emission spectrum wavelengths in the NIR between 750-1600 nm.
Lights or LEDs with emission spectrum wavelengths in the NIR between 800-900 nm.
Lights or LEDs with emission spectrum wavelengths in the NIR between 1050-1150 nm.
Lights or LEDs with infrared emission spectrum in combination of optical filters.
Light sources with coupled optical filters to restrict the radiation within the infrared spectrum, optionally motor controlled.
   Additionally, the infrared light source (4) for its application to operations of complications in monochorionic twins pregnancies is, preferably:
A monochromatic source centered between 815-835 nm, preferably centered at 821 nm. The latter value corresponds to a wavelength of optimal transmittance in the amniotic fluid.
A monochromatic source centered at 1050-1090 nm, preferably centered at 1070 nm. The latter value corresponds to a wavelength of optimal transmittance in the amniotic fluid.

The light can be coupled to the video channel of the endoscope using different optical elements (6) such as beam splitters, hot mirrors (intended as infrared-reflecting mirrors), cold mirrors (intended as visible light-reflecting mirrors), dichroic mirrors, polarizers, diffusers, diffractive optical elements, analyzers, holographic optical elements, phase plates, acusto-optic materials, dazzlers, shapers, partial mirrors, dichroic prism systems, tunable optical filters, multibifurcated light guides, polarization beam splitters or any other optical devices able to modify their transmission or reflection conditions depending on the wavelength, polarization or other optical property in order to split or combine the optical path for either or both detection and illumination, also including the encapsulation in optical fiber when the optical path is a fiber optic path.

The same channel can also be used for detection by the employment of filters (8) and lenses (9) to form the images on a video camera (CCD, CMOS, EM-CCD, etc.), in order to digitize them to be further processed by the image processing unit (2).

Additionally, an image intensifier can be added to the video cameras (10), (11) if the detected signals are very weak or they show a low quality.

In order to simplify the multimodal image acquisition unit (1), light sources (4), (5) can be coupled to the video systems (10), (11) by using two channels of the endoscope (3), as shown in FIG. 2.

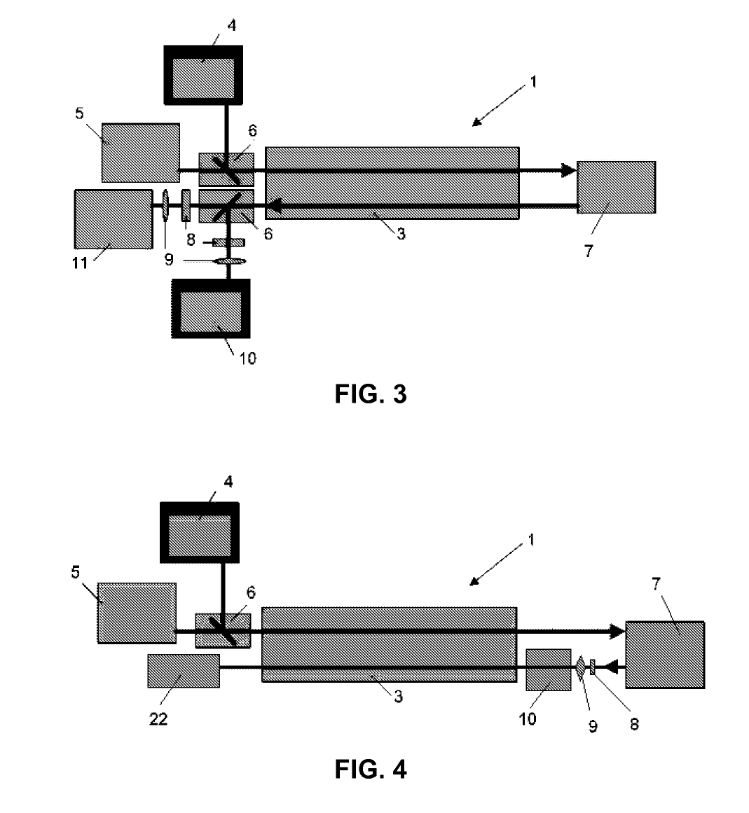

Also a separate channel can be used only for illumination, employing different optical elements (6), as shown in FIG. 3.

In another embodiment of the invention, a CCD, CMOS or EM-CCD camera (10), installed at the probe of the endoscope and coupled to an electric connection (22), is employed for the sequential detection of different bands or wavelengths sequentially emitted by the light sources (4), (5), as shown in FIG. 4. Optionally, at least one filter (8) in the camera (10) can be a color filter array (CFA) or a color filter mosaic (CFM) for the separation of one or more infrared spectral bands.

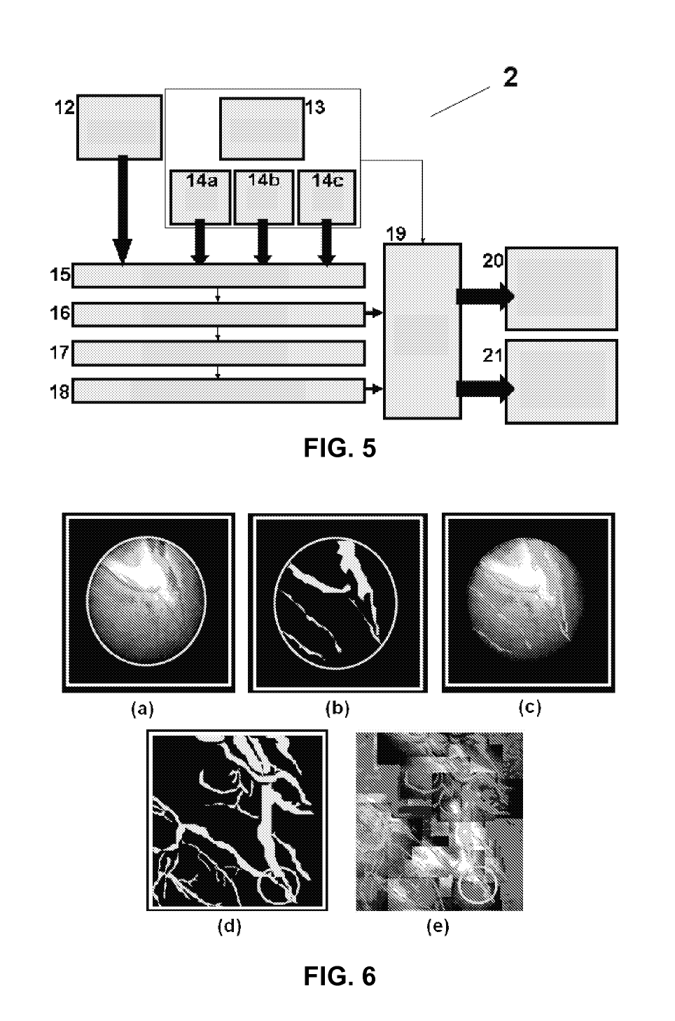

The image processing unit (2) forming part of the equipment of the present invention is a device responsible for processing and displaying the enhanced images to the surgeon in real time after having been acquired by the multimodal image acquisition unit (1). Said device comprises at least each of the methods listed below, as shown in the diagram of FIG. 5, by the implementation of the appropriate hardware and software in GPUs, FPGAs, CPU-based systems or any other hardware performing real-time processing through local, distributed or parallel computing. In FIG. 5, for better understanding, the infrared image has been referenced with (12), the visible image with (13), the reflected image in red, green and blue, with (14a), (14b) and (14c) respectively, the different methods with (15), (16), (17), (18) and (19), enhanced local display with (20) and the enhanced overall display with (21). The essential tasks that said hardware and software execute, i.e. the procedures of signal processing to improve the imaging of the equipment that makes this unit are:

Method 1. Normalization (15): signal processing procedure to normalize the amount of light that illuminates the tissue (7), by real-time comparing the intensities in each of the points in the image of the intensity of visible light (red, green and blue) and infrared light and the use of low-pass filter on the images, estimating the amount of incident infrared light in a reproducible manner.

Inputs:
Reflected red image R_{R}(x,y) (14a), wherein (x, y) refers to the two-dimensional pixel coordinates in the image obtained.
Reflected green image R_{G}(x,y) (14b).
Reflected blue image R_{B}(x,y) (14c).
Reflected infrared image R_{NIR}(x,y) (12).

Outputs:
Estimated illumination image Î_{NIR}(x,y).

Method 2. Segmentation (16): Signal processing procedure to real-time segment the blood vessel images based on spectral analysis of infrared and visible light.

Inputs:
Estimated illumination image Î_{NIR}(x,y).
Reflected infrared image R_{NIR}(x,y).

Outputs:
Blood vessel probability Pₘ (vessel|x, y), m=1, 2, . . . M. M corresponds to the different image-acquisition modes different from the RGB mode.
Blood vessel segmented image V(x,y).

Essential Steps:

1. Using the ratio of infrared light reflected and estimated incident light a probability to each point can be assigned forming a new image that contains the probability of being “blood vessel” for each point on the screen by a sigmoid curve, for example:

$${P}_{1} ( vessel \vert x , y ) = \frac{1}{1 + exp ( - a \frac{{R}_{NIR} ( x , y )}{{\hat{I}}_{NIR} ( x , y )} )} ,$$

where a is a constant manually or automatically chosen, R_{NIR}(x,y) is the infrared reflected image and Î_{NIR}(x,y) is the estimated image using method 1.
2. By low-pass filtering the probabilities, a new probability image is generated, which averages the probabilities within a neighborhood, P₂(vessel|x, y).
3. The essential steps 1 and 2 can be repeated for each of the wavelengths or optical imaging modes that are available for the multimodal imaging unit (1), thus generating a range of images of probability Pₘ(vessel|x, y) for m=1, 2 . . . M.
4. Using a threshold over Pₘ(vessel|x, y) and the application of morphologic operations, the image is segmented between “blood vessel” with a value of 1 for V(x,y) and “not blood vessel” with a value of 0 for V(x,y).
5. The incorporation of image acquisition modes in the multimodal imaging unit (1) improves the accuracy of the segmentation and/or obtains a greater number of segmented classes, such as arteries and veins using additional wavelengths, or collagen structure, by using polarizers. The latter application is particularly relevant for dermatology.

Method 3. Tracking (17): Signal processing procedure for real-time tracking and co-localizing blood vessels between two consecutive scenes from images generated by Methods 1 and 2.

Inputs:
Blood vessel probability image Pₘ(vessel|x, y), m=1, 2, . . . M.
Blood vessel segmented image V(x,y).
Previous blood vessels probability images Pₘ′(vessel|x, y), m=1, 2, . . . M.
Previous blood vessel segmented image V′(x,y) or vascular map image T(x,y).

Outputs:
Displacement vector between two images d(x,y), used for measuring displacement distances.
Cross correlation coefficient between images Cv.

Essential Steps:
Option A:
   1. A predictive model favors the blood vessels natural direction and smoothes the blood vessels edges of the previous V′(x,y) and the current V(x,y) images, resulting in Vp′(x,y) and Vp(x,y), respectively.
   2. The maximum of the normalized crossed correlation between Vp′(x,y) and Vp(x,y) is detected.
   3. The distance of the maximum to the origin of coordinates gives the displacement distance d(x,y).
   4. Cross correlation coefficient is calculated, as the maximum of the normalized cross correlation.
Option B:
   1. A predictive model which favors the blood vessels natural direction and smoothes the blood vessels edges of the previous V′(x,y) and the current V(x,y) images, resulting in Vp′(x,y) and Vp(x,y), respectively.
   2. The area which delimitates the full width half maximum of the cross correlation between Vp′(x,y) and Vp(x,y) is detected.
   3. The distance of the centroid or center of mass of the said area, weighted or not, respect to the origin gives the displacement distance d(x,y). Centroid and center of mass calculations are intended as usual image-processing operations for calculating the center of an area.
   4. The quotient of the cross correlation is the weighted average of the normalized cross correlation.
Option C:
   1. The most probable displacement is found, d(x,y), maximizing likelihood, by comparing the previous and current probability images Pₘ′(vessel|x, y) and Pₘ (vessel|x, y), respectively.
   2. The overlapping area of the previous V′(x,y) and current V(x,y) is calculated and normalized with respect to the total area of the field of view of the image, this gives Cv.

Method 4. Mapping (18): Signal processing procedure to generate the map of the anatomical structures or tissues, preferably the vascular structures in real-time, based on images and tracking coordinates obtained from methods 1 and 2.

Inputs:
Position vector p(x,y).
Displacement vector between the two images d(x,y).
Cross correlation coefficient between images Cv.
Reflected red image R_{R}(x,y) (14a).
Reflected green image R_{G}(x,y) (14b).
Reflected blue image R_{B}(x,y) (14c).

Outputs:
Vascular map image T(x,y).
Global image G(x,y,c) (Note: c refers to colors red, green, blue).
Previous blood vessels probability images Pₘ′(vessel|x,y), m=1, 2, . . . M.
Previous blood vessel segmented image V′(x,y).

Essential Steps:

These techniques are known as Stitching or Mosaicing and are used in computer vision. A possible implementation is:
1. A threshold >0.5 is applied over the cross correlation coefficient, Cv.
2a. If Cv<0.5, the automatic system assumes that the current image contains errors and does not use it for the vascular map stitching.
3a. Search the current image V(x,y) in the global vascular map T(x,y) through the Tracking algorithm (Method 3). New parameters d(x,y) and Cv are obtained.
4a. If Cv>0.5 proceed to step 2b, else skip the rest of the steps and wait until next image acquisition.
2b. If Cv>0.5, the current image V(x,y) is placed on the global image T(x,y) in a way that the previous position p(x,y) and its displacement d(x,y) is taken into account.
3b. The current image which belongs to the visible in the red reflected image R_{R}(x,y) (14a), green reflected image R_{G}(x,y) 14b and blue reflected image R_{B}(x,y) (14c) in the global image G(x,y,c) in a way that the previous position p(x,y) and its displacement d(x,y) is taken in to account, where c, for instance, refers to the color in a standard video image c=R, G or B.
4b. Prepare the system for a new iteration. Transfer the current image V(x,y) to the previous image V′(x,y), i.e., V′(x,y)=V(x,y).
5b. Transfer the current probabilities to the previous ones. Pₘ′(vessel|x, y)=Pₘ (vessel|x, y)
6b. Update the position by d(x,y) and p(x,y).

Method 5. Fusion (19): Signal processing procedure to merge in real-time the image of the visible (produced by a standard endoscope) with information from method 3.

Inputs:
Vascular map image T(x,y).
Global image G(x,y,c).
Reflected red image R_{R}(x,y) (14a).
Reflected green image R_{G}(x,y) (14b).
Reflected blue image R_{B}(x,y) (14c).
Blood vessels segmented image V(x,y).

Outputs:
Color image of local enhanced vision VEL(x,y,c).
Color image of global enhanced vision VEG(x,y,c).

Essential Steps:

1. Image VEL(x,y,c) is obtained by the weighted adding of the segmented blood vessel image V(x,y) overlapped onto one or many Visible images: reflected red image R_{R}(x,y) (14a), reflected green image R_{G}(x,y)(14b) and reflected blue image R_{B}(x,y) (14c).

2. Image VEG(x,y,c) is obtained by adding the segmented vascular map image T(x,y) overlapped onto one of the channels or colors c of the global image G(x,y,c).
3. Achieving a digital image that can be sent to one or several monitors, projectors or generic device able to represent a digital or analog image.
4. A user interface is created to choose the viewing modality to display in each of the monitors (or equivalent): VEL(x,y,c), VEG(x,y,c), V(x,y), T(x,y) or G(x,y,c).

In order to clarify the effect of the described methods, different vision modes available to the equipment described by the present invention are depicted in FIG. 6, showing (a) the vision mode offered by a standard endoscope, (b) the segmentation (16) of blood vessels through NIR analysis, (c) fusion (19) of visible and NIR images, (d) mapping (18) reconstruction and (e) mosaic reconstruction by tracking (17) of consecutive images.

To sum up, the signal processing procedure to improve infrared vision of anatomical structures with the equipment of the invention is performed in the image processing unit (2) with the specific hardware and software implemented in GPUs, FPGAs, CPU-based systems or any other hardware performing real-time processing through local, distributed or parallel computing, comprising said procedure at least the following methods:

Method 1. Normalization (15): Signal processing procedure to normalize the amount of light that illuminates the tissue (7), by real-time comparing of the intensities in each of the points in the image of the intensity of visible light (red, green and blue) and infrared light; and use of low pass filter on the images. The amount of incident infrared light is estimated in a reproducible manner.
Method 2. Segmentation (16): Signal processing procedure to segment the anatomical structures or tissues, preferably the vascular structures, based on real-time spectral analysis of infrared and visible images.
Method 3. Tracking (17): Signal processing procedure for real-time tracking and co-localization of the anatomical structures or tissues, preferably the vascular structures, between two consecutive images generated by Methods 1 and 2.
Method 4. Mapping (18): Signal processing procedure to generate the real-time map of the anatomical structures or tissues, preferably the vascular structures from the images and tracking coordinates obtained from methods 1 and 2.
Method 5. Fusion (19): Signal processing procedure to fuse the visible image (produced by a standard endoscope) with information from method 3.

The equipment can further integrate more image modes by using additional sources of light (both visible and infrared) and/or additional optical systems to acquire different imaging modes in the multimodal imaging unit (1).

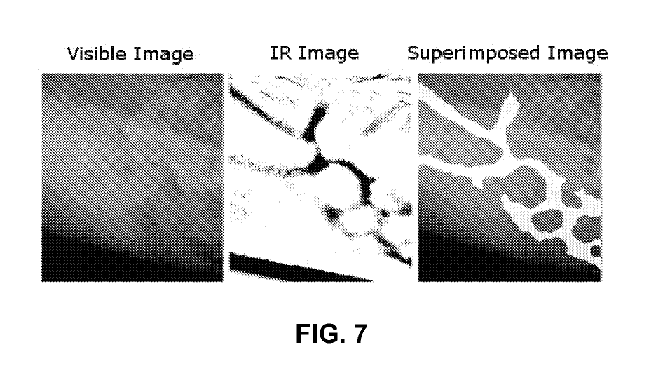

The present invention offers, additionally, relevant applications to any type of endoscopy surgery, such as gastrointestinal tract endoscopy, respiratory tract endoscopy, arthroscopy, gynecologic endoscopy, colposcopy, urologic endoscopy, otoscopy, or plastic surgery endoscopy, among others. The invention further provides applications to other medical procedures, such as skin or open surgical procedures, by the replacement of the endoscope, laparoscope or fetoscope by an optical objective (intended as a lens, a mirror or other optical instrument that gathers the light coming from the object being observed) adapted to its employment in said medical procedures. As an example, FIG. 7 shows the images obtained by the use of the techniques of vascular detection here described applied to the surface of the forearm, where visible modes are fused to the NIR image.

The disclosed invention also offers the possibility to perform functional analysis of the anatomical structures. Other modalities of the present invention offer the classification of different anatomical structures, such as collagen by the use of polarization imaging and/or second harmonic. It can also be used to distinguish between variations in the same anatomical structures to detect anomalies that lead to diagnose clinic conditions. All this automated and quantitative data acquisition is not only adaptable to the guide surgery but also to the robotized remote or automated surgery.

Having sufficiently described the nature of the present invention, as well as how to implement it, it is not considered necessary to extend the explanation for any expert in the field to understand its scope and the advantages that derive from it, but highlighting that, within its fundamental nature, it can be put into practice in other embodiments that differ in the details from that indicated though the examples, and which remain covered by the claimed protection providing that the fundamental nature is not altered, changed or modified.

DESCRIPTION OF THE NUMERICAL REFERENCES USED

| Reference | Description |
|---|---|
| (1) | Multimodal or multispectral image acquisition unit |
| (2) | Image processing unit |
| (3) | Endoscope, fetoscope or laparoscope |
| (4) | Infrared light source |
| (5) | White light source |
| (6) | Optical elements |
| (7) | Anatomical structure, tissue or vascular structure |
| (8) | Filter |
| (9) | Lens |
| (10) | Video camera |
| (11) | Video camera |
| (12) | Infrared image |
| (13) | Visible image |
| (14a) | Red image |
| (14b) | Green image |
| (14c) | Blue image |
| (15) | Normalization method |
| (16) | Segmentation method |
| (17) | Tracking method |
| (18) | Mapping method |
| (19) | Fusion method |
| (20) | Enhanced local display |
| (21) | Enhanced overall display |
| (22) | Electric connection |

1-10. (canceled)

11. Equipment for infrared-enhanced imaging and functional analysis of anatomical structures and tissues, characterized in that it comprises at least two cooperating units:
a multimodal or multispectral image acquisition unit (1) for acquiring images from the interior of the human body comprising an image capturing device, wherein said image capturing device is equipped with at least one channel from where the video images from the interior of the human body are acquired, and wherein at least one infrared light source (4) and at least one white light source (5) or a light source comprising blue, green and red wavelengths are coupled to said at least one channel.
an image processing unit (2), comprising a device with an interface for processing and displaying the enhanced images of the human body acquired by the image acquisition unit (1) and for real-time locating the position of the image capturing device, wherein said image processing unit (2) comprises at least the following signal processing means:
Normalization (15) signal processing means for normalizing the amount of light that illuminates the anatomical structures or tissues (7) by real-time comparing the intensities of visible light (red, green and blue) and infrared light in each image point, using a low-pass filter in the images and estimating the amount of incident infrared light;
Segmentation (16) signal processing means for segmenting the images of the anatomical structures or tissues based on the real-time spectral analysis of the infrared and visible light;
Tracking (17) signal processing means for obtaining tracking coordinates in real time and localizing the anatomical structures or tissues between two consecutive images generated by the normalization (15) means and the segmentation (16) means;
Mapping (18) signal processing means for generating a real-time map of the anatomical structures or tissues from the images of the anatomical structures or tissues and through the use of tracking coordinates obtained by tracking (17) means; and
Fusion (19) signal processing means for merging the visible-light images produced by the image acquisition unit (1) with the information obtained by any of the normalization (15) means, segmentation (16) means, tracking (17) means and mapping (18) means.

12. The equipment according to claim 11, wherein the anatomical structures and tissues are vascular structures such as blood vessels.

13. The equipment according to claim 11, wherein the image capturing device is an endoscopic image acquisition device comprising an endoscope, a fetoscope or a laparoscope (3).

14. The equipment according to claim 11 wherein the light sources (4, 5) are coupled to the video channel of the endoscope by means of optical elements (6) selected from the group consisting of beam splitters, hot mirrors, cold mirrors, dichroic mirrors, polarizers, diffusers, diffractive optical elements, analyzers, holographic optical elements, phase plates, acusto-optic materials, dazzlers, shapers, partial mirrors, dichroic prism systems, tunable optical filters, multibifurcated light guides, polarization beam splitters, and any other optical devices, wherein the other optical devices are able to modify their transmission or reflection conditions depending on the wavelength, polarization or other optical property in order to split or combine the optical path for detection, illumination or both detection and illumination.

15. The equipment according to claim 14 characterized in that the same channel in the image capturing device is employed for detection of anatomical structures by the use of optical elements (6), optionally in combination with filters (8) and lenses (9), in order to form images in one or more video cameras (10), (11) selected from the group consisting of CCD, CMOS, and EM-CCD cameras and to digitize said images for its further processing by the image processing unit (2).

16. The equipment according to claim 15, further comprising image intensifiers provided to the video cameras (10), (11).

17. The equipment according to claim 11, characterized in that at least an infrared light source (4) and at least one white light source (5) are coupled to video cameras (10), (11) by using two channels of the image capturing device.

18. The equipment according to claim 11 characterized in that the at least one infrared light source (4) and the at least one white light source (5) or a light source comprising blue, green and red wavelengths, are comprised in a channel used only for the illumination in combination with optical elements (6), wherein the equipment is thus configured with a channel used only for the illumination and a channel used only for the detection, optionally further comprising additional optical elements such as filters (8) and lenses (9).

19. The equipment according to claim 11, further comprising a CCD, CMOS or EM-CCD camera (10) installed at the distal probe of the image capturing device and coupled to an electric connection (22), wherein the camera (10) is employed for the sequential detection of different bands or wavelengths sequentially emitted by the light sources (4), (5), wherein at least one filter (8) in the camera (10) can optionally be a color filter array or a color filter mosaic for the separation of one or more infrared spectral bands.

20. The equipment according to claim 11 wherein the image acquisition unit (1) comprises as image capturing device an optical objective adapted to skin and open surgical procedures.

21. A procedure of signal processing of images of anatomical structures and tissues, characterized in that said anatomical structures or tissues are illuminated by at least one infrared light source (4) and at least one white light source (5) or a light source comprising blue, green and red wavelengths, said method comprising implementing in the image processing unit (2) at least:
normalizing (15), consisting of signal processing to normalize the amount of light that illuminates the tissue (7) by means of the real-time comparison of the intensities of visible light (red, green and blue) and infrared light in each image point and the use of a low-pass filter in the images;
segmenting (16), consisting of signal processing to segment the images of the anatomical structures or tissues based on the real-time spectral or multimodal analysis of the infrared and visible light;
Tracking (17), consisting of signal processing to obtain tracking coordinates in real time and to track and localize the anatomical structures or tissues between two consecutive images generated by normalization (15) and segmentation (16);
Mapping (18), consisting of signal processing to generate a real-time map of the anatomical structures or tissues from the images of the anatomical structures or tissues and through the use of tracking coordinates obtained by tracking (17); and
Fusion (19), consisting of signal processing to merge visible-light images produced by the image acquisition unit (1) with the information obtained by any of the methods corresponding to normalization (15), segmentation (16), tracking (17) and mapping (18).
by GPUs, FPGAs, CPU-based systems or any other hardware performing real-time processing through local, distributed or parallel computing.

22. An image processing unit (2) comprising a device with an interface to process and display the enhanced images of anatomical structures and tissues illuminated by at least one infrared light source (4) and at least one white light source (5) or a light source comprising blue, green and red wavelengths, wherein said image processing unit (2) comprises at least the following signal processing means:
Normalization (15) signal processing means for normalizing the amount of light that illuminates the anatomical structures or tissues (7) by real-time comparing the intensities of visible light (red, green and blue) and infrared light in each image point, using a low-pass filter in the images and estimating the amount of incident infrared light;
Segmentation (16) signal processing means for segmenting the images of the anatomical structures or tissues based on the real-time spectral analysis of the infrared and visible light;
Tracking (17) signal processing means for obtaining tracking coordinates in real time to localize the anatomical structures or tissues between two consecutive images generated by the normalization (15) means and the segmentation (16) means;
Mapping (18) signal processing means for generating a real-time map of the anatomical structures or tissues from the images of the anatomical structures or tissues and through the use of tracking coordinates obtained by tracking (17) means; and
Fusion (19) signal processing means for merging the visible-light images produced by the image acquisition unit (1) with the information obtained by any of the normalization (15) means, segmentation (16) means, tracking (17) means and mapping (18) means.

23. The procedure of claim 21 for use in treatments of monochorionic twins pregnancies, discordant malformations and selective intrauterine growth restriction operations.

24. The procedure of claim 21 for application in endoscopy surgery procedures.

25. The procedure of claim 24, wherein the endoscopy surgery procedures are selected from the group consisting of gastrointestinal tract endoscopy, respiratory tract endoscopy, arthroscopy, gynecologic endoscopy, colposcopy, urologic endoscopy, otoscopy, and plastic surgery endoscopy.

26. The procedure of claim 21, for application in skin and open surgical procedures.

27. The procedure of claim 21, to report functional information on the anatomical structures or to assess the collagen structure of the tissues.

28. The procedure of claim 27, wherein the functional information on the anatomical structures is the amount of oxygen level in tissues or vessels to distinguish between arteries and veins.
